SEQUENCE LISTING (7 sequences)

SEQ ID NO: 1 - protein, 247 aa, Homo sapiens
APSSDSSLSSAPLPEYSSCQPPSAPPPSYAKVISAPVSDATPDYAVVTALPPTSTPPTPPLRHAATRFATSLGSAFHPVLPHYATVPRPLNKNSRPSSPVNTPSSQPPAAKSCAQPTSNFSPLPPSPPIMISSPPGKATGPRPVLPVCVSSPVPQMPPSPTAPNGSLDSVTYPVSPPPTSGPAAPPPPPPPPPPPPPPPLPPPPLPPLASLSHCSGSQASPPPGTPLASTPSSKPSVLPSPSAGAPA

SEQ ID NO: 2 - protein, 4 aa, Homo sapiens
FYLG

SEQ ID NO: 3 - protein, 19 aa, Homo sapiens
AQSKVTATQDSTNLRCIFC

SEQ ID NO: 4 - protein, 21 aa, Homo sapiens
RDSPRKNQIVFDNRSYDSLHR

SEQ ID NO: 5 - DNA, 12 bp, Homo sapiens
ttctatttaggg

SEQ ID NO: 6 - DNA, 57 bp, Homo sapiens
gcccagagcaaggttactgctacccaggacagcactaatttgcgatgtattttctgt

SEQ ID NO: 7 - DNA, 63 bp, Homo sapiens
acgggattctccaaggaaaaatcagattgtttttgacaacaggtcctatgattcattacacag

(19) United States
(12) Patent Application Publication
(10) Pub. No.: US 2013/0004424 A1
(43) Pub. Date: Jan. 3, 2013

(54) METHODS FOR DETERMINING AGENTS TARGETING MENA ISOFORMS AND USES THEREOF FOR DIAGNOSIS AND TREATMENT OF METASTATIC TUMORS

(76) Inventors: Frank Gertler, Boston, MA (US); John Condeelis, Bronx, NY (US)

(21) Appl. No.: 13/575,359

(22) PCT Filed: Jan. 19, 2011
(86) PCT No.: PCT/US11/00095

§ 371 Date: Sep. 14, 2012

Related U.S. Application Data
(60) Provisional application No. 61/336,929, filed on Jan. 27, 2010.

(51) Int. Cl. C12Q 1/02; G01N 33/566; A61P 35/00; A61K 49/00; A61K 39/395; A61K 38/02; C12Q 1/68; G01N 33/574

(52) U.S. Cl. 424/9.1; 435/29; 435/6.13; 435/7.9; 436/501; 435/6.11; 435/7.1; 424/174.1; 514/19.3

(57) ABSTRACT

The present invention relates to methods of determining agents that inhibit Mena⁺ or Mena^{INV/+}, and uses of agents that bind to and/or inhibit Mena⁺ or Mena^{INV/+} for diagnosis and treatment of metastatic tumors.

### CROSS-REFERENCE TO RELATED APPLICATION

This application claims the benefit of U.S. Provisional Patent Application No. 61/336,929, filed Jan. 27, 2010, the content of which is hereby incorporated by reference into the subject application.

### STATEMENT OF GOVERNMENT SUPPORT

This invention was made with government support under grant numbers 1-U54-CA112967, GM58801, GM38511, CA100324, and CA113395 awarded by the National Institutes of Health, U.S. Department of Health and Human Services. The government has certain rights in the invention.

### FIELD OF THE INVENTION

The present invention relates to methods of determining and using agents that inhibit Mena⁺ or Mena^{INV/+} for diagnosis and treatment of metastatic tumors.

### BACKGROUND OF THE INVENTION

Throughout this application various publications are referred to in parenthesis. Full citations for these references may be found at the end of the specification. The disclosures of these publications are hereby incorporated by reference in their entirety into the subject application to more fully describe the art to which the subject invention pertains.

Mena is a cytoskeletal protein and is a member of the Ena/VASP family of proteins. These proteins are regulatory molecules which control cell movement, motility and shape in a number of cell types and organisms. They prevent the actin filaments from being capped by capping proteins at their barbed ends, amplifying the barbed end output and increasing metastatic potential in many tumors. Ena/VASP proteins are also constituents of the adherence junctions necessary to seal membranes in the epithelial sheet and control actin organization on cadherin adhesion contact. This process is frequently perturbed in cancer. Mena is upregulated in mouse and rat invasive breast cancer cells and overexpressed in human breast, colon, pancreatic, cervical and lung cancers. There are a number of isoforms, or splice variants, of Mena which are differentially expressed in primary tumors, invasive cancer cells and metastases (1, 2). The broadly expressed form of the protein is referred to as “Mena”. Mena^{11a} includes the “11a” exon and is found in primary tumors and sometimes in metastases, but not in invasive and metastatic cells during dissemination as Mena^{11a} is downregulated in invasive tumor cells (1). Mena⁺ is the Mena with the “+” exon. Mena^{INV/+} has both the “+” exon and the “+++” exon (the “+++” exon is referred to here as the “INV” exon). Mena^{INV} is expressed only in invasive and metastatic cancer cells and not in primary tumors (1-3). Mena^{INV} is not observed in cells of adult animals that are not invasive cancer cells. Additionally, the “INV” exon sequence has no similarity to publicly known molecules. The presence of a hydrophobic cluster of residues in the C-terminal part of the sequence allows for a putative agent that is hydrophobic and membrane permeable. In adult animals, the “+” exon is normally only found in the central nervous system and cancer cells.

Expression of Mena^{INV} in mammary caricinoma cells increases lung metastases after injection into mammary fat pads. Mena^{INV}-cells exhibit increased in vivo cell motility rates and enhanced epidermal growth factor (EGF) chemotactic responses both in vivo and in vitro (3, 4). The Mena^{INV} cells also exhibit resistance to the epidermal growth factor receptor (EGFR) inhibitor TARCEVA® (erlotinib) both in vitro and in vivo. Compared to controls, cells expressing Mena^{INV} or Mena^{INV/+} are capable of responding to 40- or 250-fold lower EGF concentrations, respectively (3). Conversely, cells expressing Mena^{11a} exhibit reduced responses to EGF both in vitro and in vivo (3). Therefore, switching of Mena isoforms results in dramatically altered motility responses to EGF and represents a mechanism that changes the sensitivity of invasive tumor cells to inhibitors of EGFR signaling (3). Analysis of signaling pathways downstream of EGFR indicates that canonical targets, such AKT and Erk and others involved in EGF-dependent proliferation are not affected by the Mena isoforms. Therefore, the effect of Mena isoforms on EGFR responses involves non-cannoncial pathways related to motility, chemotaxis and metastasis (3, 4). The risk of tumor cells undergoing metastasis increases with an increase in density of occurrences of an endothelial cell, a macrophage, and an invasive tumor cell in direct apposition in the tumor (4).

Once tumor cells have metastasized and established secondary tumors, survival rate decreases. Therefore, a metastasis inhibitor is sorely needed. The present invention advances this need by providing a method of determining chemotherapeutic agents that target Mena^{INV} and Mena^{INV/+}.

### SUMMARY OF THE INVENTION

The present invention provides a method for determining a putative agent that binds to Mena⁺ or Mena^{INV/+}, the method comprising the steps of contacting Mena+ or Mena^{INV/+} with the putative agent and measuring bound or unbound Mena⁺ or Mena^{INV/+}.

The present invention also provides a method for determining a putative agent that inhibits Mena⁺ or Mena^{INV/+}, the method comprising the steps of contacting tumor cells expressing Mena⁺ or Mena^{INV/+} with the putative agent in the presence of a receptor tyrosine kinase-substrate gradient, and measuring actin polymerization or cell protrusion activity, wherein a decrease in or absence of actin polymerization or cell protrusion activity indicates inhibition of Mena⁺ or Mena^{INV/+}.

The present invention further provides a method for determining a putative agent that inhibits metastasis of tumor cells expressing Mena⁺ or Mena^{INV/+} in vivo, the method comprising contacting the Mena⁺ or Mena^{INV/+} expressing tumor with the putative agent, and measuring tumor metastasis.

The present invention additionally provides a method of treating a subject with a tumor expressing Mena⁺ or Mena^{INV/+}, the method comprising administering to the subject a Mena⁺ or Mena^{INV/+} inhibitor in an amount effective to treat the tumor.

The present invention also provides a method for determining a putative agent that inhibits metastasis of a tumor, the method comprising contacting the putative agent with a cell line or tissue culture that expresses Mena⁺ or Mena^{INV/+}, wherein reduction in the expression of Mena⁺ or Mena^{INV/+} is indicative that the putative agent is a candidate for inhibiting metastasis of a tumor or wherein lack of reduction in the expression of Mena⁺ or Mena^{INV/+} is indicative that the compound is not a candidate compound for inhibiting metastasis of a tumor.

The present invention provides the putative agent identified by the method for (1) determining a putative agent that binds to Mena⁺ or Mena^{INV/+}, the method comprising the steps of contacting Mena⁺ or Mena and measuring bound or unbound Mena⁺ or Mena^{INV/+}; (2) determining a putative agent that inhibits Mena⁺ or Mena^{INV/+}, the method comprising the steps of contacting tumor cells expressing Mena⁺ or Mena^{INV/+} with the putative agent in the presence of a receptor tyrosine kinase-substrate gradient, and measuring actin polymerization or cell protrusion activity, wherein a decrease in or absence of actin polymerization or cell protrusion activity indicates inhibition of Mena⁺ or Mena^{INV/+}; or (3) determining a putative agent that inhibits metastasis of tumor cells expressing Mena⁺ or Mena^{INV/+} in vivo, the method comprising contacting the Mena⁺ or Mena^{INV/+} expressing tumor with the putative agent, and measuring tumor metastasis.

The present invention provides a pharmaceutical composition comprising a Mena⁺ or Mena^{INV/+} inhibitor formulated in dosage form for treating a tumor.

The present invention also provides the use of a Mena⁺ or Mena^{INV/+} inhibitor for the treatment of a tumor. The present invention further provides the use of a Mena⁺ or Mena^{INV/+} inhibitor for the preparation of a medicament for the treatment of a tumor.

### BRIEF DESCRIPTION OF THE DRAWINGS

FIG. 1A-1B. (A) Mena sequence showing location of INV and + exons. (B) In vitro sensitivity of Mena, Mena^{INV}, Mena^{INV/+} and GFP control to various EGF concentrations. Protrusion of starved cells 3 minutes after EGF addition. GFP control expresses no MENA isoform. Control drops to background level around 0.5 nM. MENA^{INV/+} can respond to 2 orders of magnitude lower EGF concentration, compared to control. *p<0.05 vs. GFP control.

FIG. 2A-2B. Mena^{INV} promotes invasion of cells in vivo assay. (A) Isoform of Mena expressed affects response to EGF gradient. Optimal response of each isoform is dependent on EGF concentration. Mena^{INV} response peaks around 1 nM, Mena response peaks around 25 nM. (B) Graphic of in vivo assay with needle collection. Primary tumor may be xenograft.

FIG. 3A-3C. Mena^{INV} cells are less sensitive to inhibition by erlotinib and still participate in the paracrine loop during in vivo invasion. Collection needle contained both EGF and erlotinib. MTLn3-EGFP cells are control. **p<0.01, ***p<0.001.

FIG. 4A-4D. Mena isoform containing both + and INV exons in the same transcript is expressed in needle collected cells from PyMT tumor model (1). (A) Diagram of Mena domain organization with primers. (B-D) APTC—average primary tumor cells. NC—needle collected cells. +plasm—plasmid of the Mena+exon. +++plasm—exon of the Mena INV exon.

FIG. 5. Kaplan Meir survival curves of PyMT Mena transgenic animals.

FIG. 6. In vivo invasion assay of PyMT mena transgenic animals. **p<0.01, ***p<0.005.

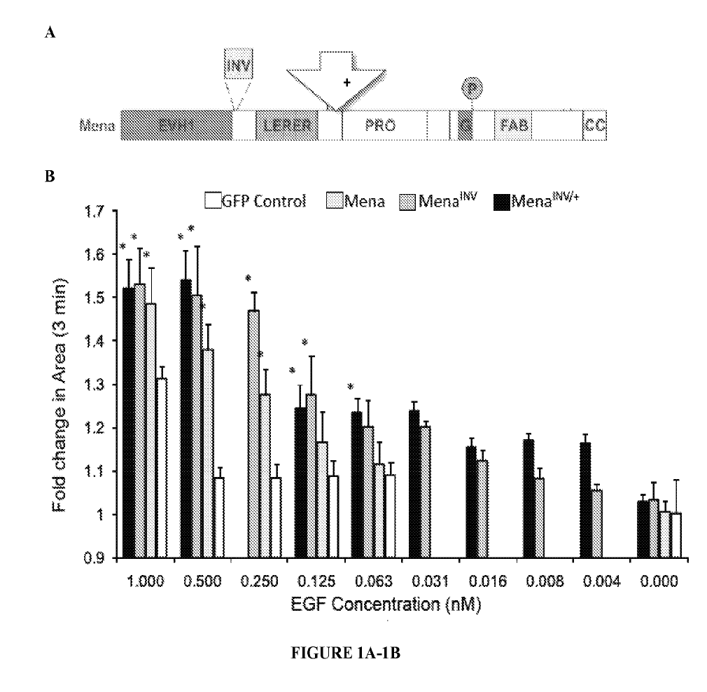

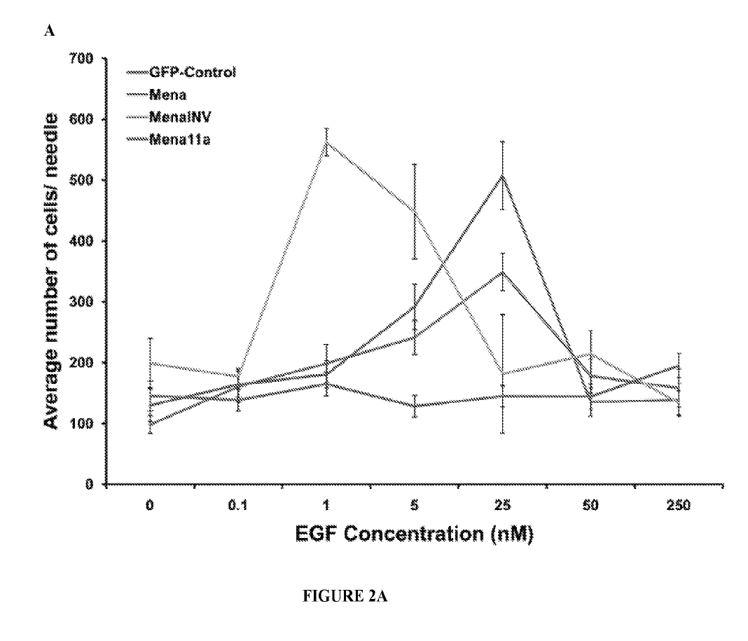

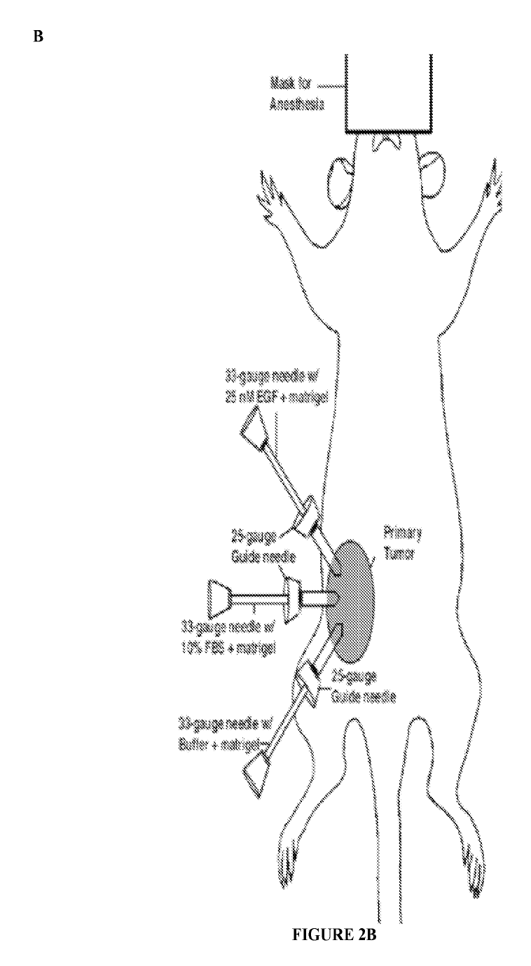

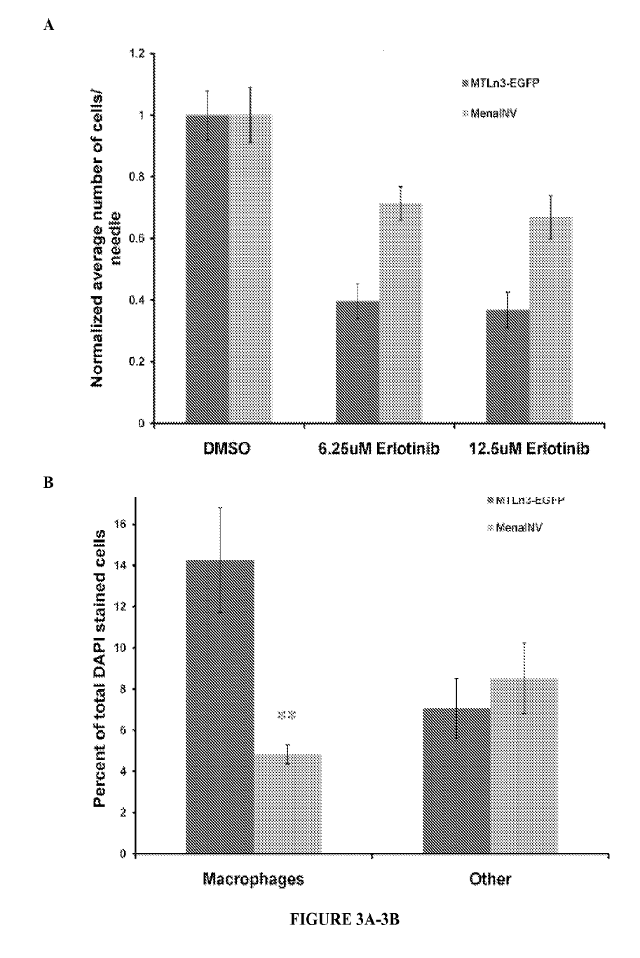

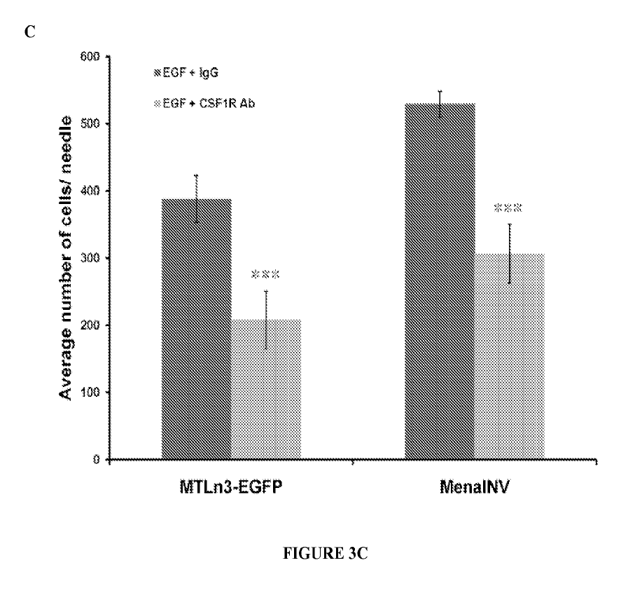

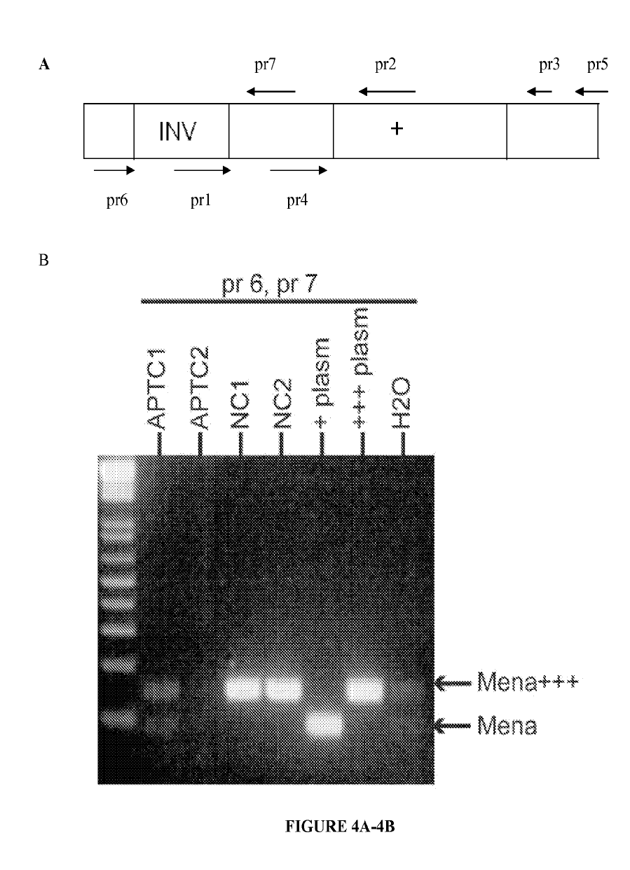

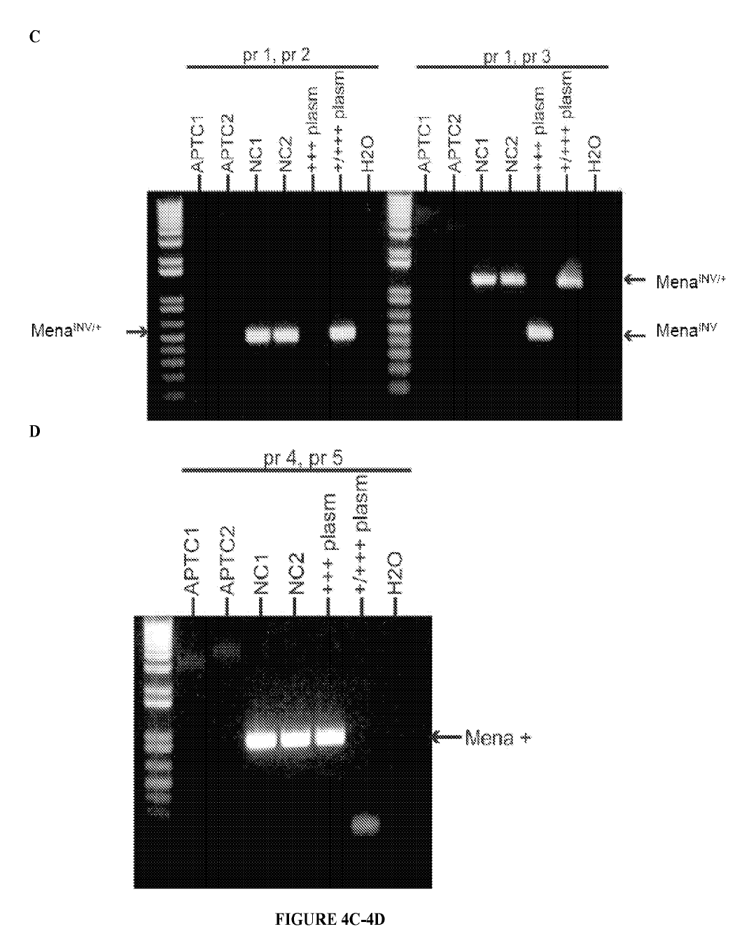

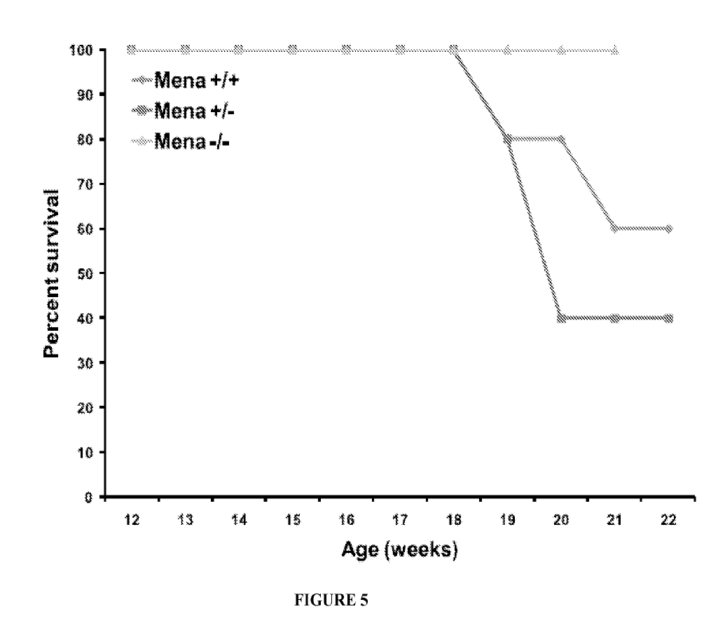

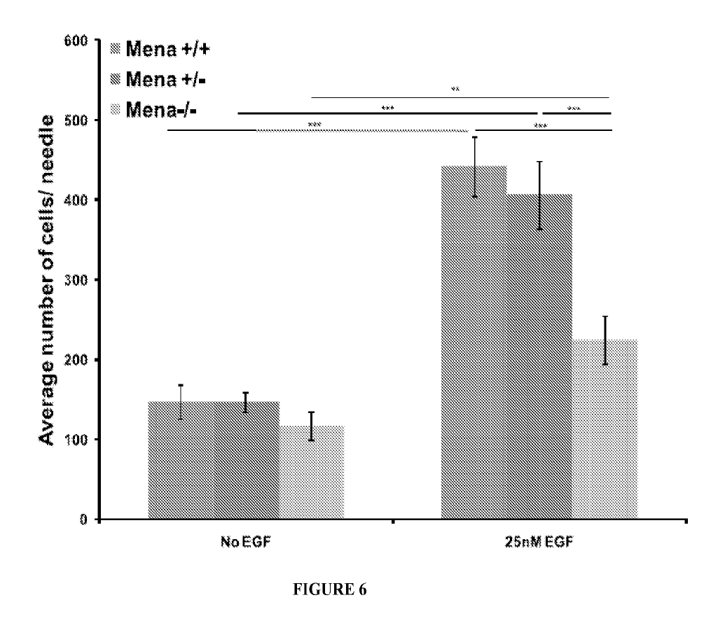

### DETAILED DESCRIPTION OF THE INVENTION

The present invention provides a method for determining a putative agent that binds to Mena⁺ or Mena^{INV/+}, the method comprising the steps of contacting Mena⁺ or Mena^{INV/+} with the putative agent and measuring bound or unbound Mena⁺ or Mena^{INV/+}, wherein a increase in Mena⁺ or Mena^{INV/+} bound to the agent or a decrease in unbound Mena⁺ or Mena^{INV/+} in the presence of the agent indicates that the agent binds to Mena⁺ or Mena^{INV/+}. Mena⁺ or Mena^{INV/+} for example, can be applied to an artificial substrate to screen for agents that bind to these Mena isoforms in vitro.

The present invention also provides a method for determining a putative agent that inhibits Mena⁺ or Mena^{INV/+}, the method comprising the steps of contacting tumor cells expressing Mena⁺ or Mena^{INV/+} with the putative agent in the presence of a receptor tyrosine kinase-substrate gradient, and measuring actin polymerization or cell protrusion activity, wherein a decrease in or absence of actin polymerization or cell protrusion activity in the presence of the agent indicates inhibition of Mena⁺ or Mena^{INV/+} and wherein a lack of decrease in actin polymerization or cell protrusion activity indicates lack of inhibition of Mena⁺ or Mena^{INV/+}.

Tumor cells expressing Mena⁺ or Mena^{INV/+} can be used to screen for agents that inhibit the ability of Mena⁺ or Mena^{INV/+} to sensitize tumor cells to low concentrations of epidermal growth factor (EGF) that, in the absence of these Mena isoforms, do not normally stimulate action polymerization and/or cell protrusion activity.

Cells expressing the different Mena isoforms can be used to identify inhibitors of EGF-dependent motility/chemotaxis and dissemination of tumor cells from the primary tumor. Expression of Mena^{INV} or Mena^{INV/+} renders cells refractory to TARCEVA® (erlotinib) and other EGFR inhibitors while potentiating their response to EGF. Therefore, cells expressing the different Mena isoforms can be used to screen for inhibitors that target the components of the EGF response that are specific to motility and metastasis-related responses. Such a screen can use the increased lamellipodial protrusion or enhanced actin polymerization observed in cells expressing Mena^{INV} or Mena^{INV/+} after treatment with low EGF concentrations. Differential mass spectrometry approaches such as SILAC may be used to compare Mena^{INV} or Mena^{INV/+} with Mena11a to identify targets that distinguish between the enhanced or suppressed EGF responses in cells expressing different Mena isoforms.

The present invention additionally provides a method for determining a putative agent that inhibits metastasis of a tumor, the method comprising contacting the putative agent with a cell line or tissue culture that expresses Mena⁺ or Mena^{INV/+}, wherein reduction in the expression of Mena⁺ or Mena^{INV/+} in the presence of the agent is indicative that the putative agent is a candidate for inhibiting metastasis of a tumor or wherein lack of reduction in the expression of Mena⁺ or Mena^{INV/+} is indicative that the agent is not a candidate for inhibiting metastasis of a tumor.

The present invention further provides a method for determining a putative agent that inhibits metastasis of tumor cells expressing Mena⁺ or Mena^{INV/+} in vivo, the method comprising contacting the Mena⁺ or Mena^{INV/+} expressing tumor with the putative agent, and measuring tumor metastasis. Tumor cells expressing Mena⁺ or Mena^{INV/+} can be transplanted into experimental animals, such as for example mice, to form tumors. Agents can be screened for their ability to inhibit metastasis from these tumors.

The invention provides a method for determining whether a subject has a metastatic tumor comprising assaying a blood, tissue and/or tumor sample of the subject for expression of Mena⁺ and/or Mena^{INV/+}, wherein overexpression of Mena⁺ and/or Mena^{INV/+} indicates the presence of a metastatic tumor.

The invention also provides a method for determining whether a subject has a metastatic tumor comprising assaying a blood, tissue and/or tumor sample of the subject for expression of Mena11a, and Mena⁺ and/or Mena^{INV/+}, wherein overexpression of Mena⁺ and/or Mena^{INV/+} and decreased expression of Mena11a together indicates the presence of a metastatic tumor.

The invention provides a method for assessing the efficacy of therapy to treat a metastatic tumor in a subject who has undergone or is undergoing treatment for a metastatic tumor, the method comprising assaying a blood, tissue and/or tumor sample of the subject for expression of Mena⁺ and/or Mena^{INV/+}, wherein overexpression of Mena⁺ and/or Mena^{INV/+} is indicative of a need to continue therapy to treat the tumor.

The invention also provides a method for assessing the efficacy of therapy to treat a metastatic tumor in a subject who has undergone or is undergoing treatment for a metastatic tumor, the method comprising assaying a blood, tissue and/or tumor sample of the subject for expression of Mena11a, and Mena⁺ and/or Mena^{INV/+}, wherein overexpression of Mena⁺ and/or Mena^{INV/+} and decrease in expression of Mena11a is indicative of a need to continue therapy to treat the tumor.

The invention further provides a method for assessing the prognosis of a subject who has a metastatic tumor, comprising assaying a blood, tissue and/or tumor sample of the subject for expression of Mena⁺ and/or Mena^{INV/+}, wherein the subject's prognosis improves with a decrease in expression of Mena⁺ and/or Mena^{INV/+}.

The invention further also provides a method for assessing the prognosis of a subject who has a metastatic tumor, comprising assaying a blood, tissue and/or tumor sample of the subject for expression of Mena11a, and Mena⁺ and/or Mena^{INV/+}, wherein the subject's prognosis improves with a decrease in expression of Mena⁺ and/or Mena^{INV/+}, and an increase in expression of Mena11a.

As used herein, changes in the expression of Mena⁺, Mena^{INV/+} and Mena11a mean changes in expression relative to their levels in normal tissue or relative to their levels in in situ (non-metastatic) carcinomas. The expression of Mena⁺, Mena^{INV/+} and Mena11a can be normalized relative to the expression of protein variants that are not changed in expression in a metastatic tumor. Examples of proteins that could be used as controls include those of the EnaNASP family that are unchanged in their expression in metastatic cells, including the 140K and 80K isoforms of Mena, and VASP. Other examples of proteins or genes that could be used as controls include those listed as relatively unchanged in expression such as N-WASP, Rac 1, Pak 1, and PKCalpha and beta. Preferred controls include the 80K and 140K isoforms of Mena and VASP. The expression of Mena⁺ or Mena^{INV/+} can be compared to expression of Mena11a, i.e. Mena+/Mena11a expression ratio or Mena^{INV/+}/Mena11a expression ratio.

The expression of Mena⁺, Mena^{INV/+} and Mena11a may be detected in vitro or in vivo. The expression may be detected at the level of the nucleic acid variant and/or at the level of the protein isoform. Where expression is detected in vitro, a sample of blood, tumor, tissue or cells from the subject may be removed using standard procedures, including biopsy and aspiration. Cells which are removed from the subject may be analyzed using immunocytofluorometry (FACS analysis). The expression of Mena⁺, Mena^{INV/+} and Mena11a may be detected by detection methods readily determined from the known art, including, without limitation, immunological techniques such as Western blotting, hybridization analysis, fluorescence imaging techniques, and/or radiation detection.

The invention provides a method of inhibiting metastasis of a tumor in a subject, the method comprising reducing the presence or activity of Mena⁺ or Mena^{INV/+} in the subject. The invention also provides a method of inhibiting metastasis of a tumor in a subject, the method comprising reducing the presence or activity of Mena⁺ or Mena^{INV/+}, and increasing the presence or activity of Mena11a in the subject.

The methods can involve intervention at the level of DNA, RNA, and/or protein. For example, the presence or activity of the isoform can be reduced by addition of an antisense molecule, a ribozyme, or an RNA interference (RNAi) molecule to the tumor, where the antisense molecule, ribozyme or RNAi molecule specifically inhibits expression of the isoform. The antisense molecule, ribozyme, or RNAi molecule can be comprised of nucleic acid (e.g., DNA or RNA) or nucleic acid mimetics (e.g., phosphorothionate mimetics) as are known in the art. Methods for treating tissue with these compositions are also known in the art. The antisense molecule, ribozyme or RNAi molecule can be added directly to the cancerous tissue in a pharmaceutical composition that preferably comprises an excipient that enhances penetration of the antisense molecule, ribozyme or RNAi molecule into the cells of the tissue. The antisense molecule, ribozyme or RNAi can be expressed from a vector that is transfected into the cancerous tissue. Such vectors are known in the art.

The presence or activity of the isoform can be reduced by addition of an antibody or aptamer to the tissue, wherein the antibody or aptamer specifically binds to and reduces the activity of the isoform in the tissue. The antibody or aptamer can be added directly to the tissue, preferably in a pharmaceutical composition comprising an agent that enhances penetration of the antibody or aptamer into the tissue. The antibody or aptamer can be encoded on a vector that is used to transfect the cancerous tissue.

The invention provides kits for detecting the presence or absence of a metastatic tumor, where the kits comprise an antibody, a peptide or an aptamer that specifically binds to Mena⁺ or Mena^{INV/+} isoforms, and/or a probe or PCR primers that specifically hybridize to nucleic acid encoding the Mena⁺ or Mena^{INV/+} isoforms. The kits can additionally comprise an agent for detecting the presence or absence of Mena11a.

TABLE 1
Human Mena Sequences

Mena+
APSSDSSLSS APLPEYSSCQ PPSAPPPSYA KVISAPVSDA TPDYAVVTAL
PPTSTPPTPP LRHAATRFAT SLGSAFHPVL PHYATVPRPL NKNSRPSSPV
NTPSSQPPAA KSCAQPTSNF SPLPPSPPIM ISSPPGKATG PRPVLPVCVS
SPVPQMPPSP TAPNGSLDSV TYPVSPPPTS GPAAPPPPPP PPPPPPPPPL
PPPPLPPLAS LSHCSGSQASP PPGTPLASTP SSKPSVLPSP SAGAPA
(SEQ ID NO: 1)
Mena ++
FYLG
(SEQ ID NO: 2)
ttctatttag gg
(SEQ ID NO: 5)
Mena^{INV} (Mena +++)
AQSKVTATQD STNLRCIFC
(SEQ ID NO: 3)
gcccagagca aggttactgc tacccaggac agcactaatt tgcgatgtat tttctgt
(SEQ ID NO: 6)
Mena 11a
RDSPRKNQIV FDNRSYDSLH R
(SEQ ID NO: 4)
acgggattct ccaaggaaaa atcagattgt ttttgacaac aggtcctatg attcattaca cag
(SEQ ID NO: 7)

An agent that specifically binds to Mena⁺ or Mena^{INV/+} or Mena11a can be labeled with a detectable marker. Labeling may be accomplished using one of a variety of labeling techniques, including peroxidase, chemiluminescent, and/or radioactive labels known in the art. The detectable marker may be, for example, a nonradioactive or fluorescent marker, such as biotin, fluorescein (FITC), acridine, cholesterol, or carboxy-X-rhodamine, which can be detected using fluorescence and other imaging techniques readily known in the art. Alternatively, the detectable marker may be a radioactive marker, including, for example, a radioisotope. The radioisotope may be any isotope that emits detectable radiation, such as, for example, ³⁵S, ³²P, or ³H. Radioactivity emitted by the radioisotope can be detected by techniques well known in the art. For example, gamma emission from the radioisotope may be detected using gamma imaging techniques, particularly scintigraphic imaging.

The expression of Mena⁺ or Mena^{INV/+} or Mena11a in a subject may be detected through hybridization analysis of nucleic acid extracted from a blood, tumor, tissue or cell sample from the subject using one or more nucleic acid probes which specifically hybridize to nucleic acid encoding Mena⁺ or Mena^{INV/+} or Menalla. The nucleic acid probes may be DNA or RNA, and may vary in length from about 8 nucleotides to the entire length of the ++ or +++ nucleic acid variant of Mena. Hybridization techniques are well known in the art. The probes may be prepared by a variety of techniques known to those skilled in the art, including, without limitation, restriction enzyme digestion of Mena nucleic acid; and automated synthesis of oligonucleotides whose sequence corresponds to selected portions of the nucleotide sequence of the Mena nucleic acid, using commercially-available oligonucleotide synthesizers, such as the Applied Biosystems Model 392 DNA/RNA synthesizer.

The nucleic acid probes may be labeled with one or more detectable markers. Labeling of the nucleic acid probes may be accomplished using a number of methods known in the art (e.g., nick translation, end labeling, fill-in end labeling, polynucleotide kinase exchange reaction, random priming, or SP6 polymerase) with a variety of labels (e.g., radioactive labels, such as ³⁵S, ³²P, or ³H, or nonradioactive labels, such as biotin, fluorescein (FITC), acridine, cholesterol, or carboxy-X-rhodamine (ROX)).

A putative agent that binds to Mena⁺ or Mena^{INV/+} protein is likely to be a Mena⁺ or Mena^{INV/+} inhibitor when administered to cancer cells. Therefore, inhibitors of the Mena isoforms will inhibit receptor tyrosine kinase-substrate-dependent motility/chemotaxis of cancer cells expressing the Mena isoforms and will inhibit dissemination of tumor cells from the primary tumor.

Receptor tyrosine kinases are high affinity cell surface receptors for many polypeptides, growth factors, and hormones. Receptor tyrosine kinase-substrates include many polypeptides, growth factors and hormones known in the art. Any of the receptor tyrosine kinase-substrates known in the art may be used to create a chemotactic gradient. For example, EGF or PDGF may be used to establish a chemotactic grandient to stimulate cell motility and chemotaxis.

Actin polymerization is necessary in chemotaxis and cytokinesis. A chemical gradient, such as a receptor tyrosine kinase-substrate gradient, results in the polymerization of actin filaments within eukaryote cells. The actin filaments are polymerized with the barbed/growing ends of the actin polymerizing towards the chemical gradient. Mena⁺ or Mena^{INV/+} prevent the capping of the growing actin polymer chains, leading to continued actin polymerization. Actin polymerization results in the creation of cell protrusions, eventually resulting in chemotaxis. Preventing the capping of actin filaments results in heightened cell protrusion activity and therefore, heightened cell motility/metastasis.

Expression of Mena⁺ or Mena^{INV/+} renders cells refractory to TARCEVA® (erlotinib) and other EGFR inhibitors while potentiating their response to EGF. Mena^{INV/+} cells respond to EGF concentrations two orders of magnitude lower than cells which do not express Mena^{INV/+}. Cells that express Mena⁺ or Mena^{INV/+} will chemotax up an EGF gradient. Since Mena⁺ or Mena^{INV/+} potentiates the cell's response to EGF, cancer cells expressing Mena⁺ or Mena^{INV/+} will respond to EGF gradients that cells which are not expressing Mena⁺ or Mena^{INV/+} will not respond to.

Actin polymerization or cell protrusion activity can be measured by any method known in the art including, but not limited to, microscopy, molecular imaging, and live cell imaging. A decrease in the level of actin polymerization or cell protrusion activity of the cancer cells in the presence of an EGF gradient after contacting the cells with the putative agent compared with the level of actin polymerization or cell protrusion activity of the cancer cells in the presence of an EGF gradient before contacting the cells with the putative agent indicates that the putative agent is an inhibitor of Mena⁺ or Mena^{INV/+}. A decrease in the level of actin polymerization or cell protrusion activity of the cancer cells is any decrease, from a statistically significant lessening in the level of actin polymerization or cell protrusion activity through a total absence of actin polymerization or cell protrusion activity after contacting the cells with the putative agent. Actin polymerization or cell protrusion activity can also be measured in vitro by any method known in the art including, but not limited to, assaying invasion of cells in a collagen gel.

The method for determining a putative agent that inhibits Mena⁺ or Mena^{INV/+} may further comprise at least one of the following controls: (1) measuring actin polymerization or cell protrusion activity of cancer cells expressing Mena⁺ or Mena^{INV/+} in the presence of an EGF gradient; or (2) contacting cells expressing Mena⁺ or Mena with the putative agent in the presence of an EGF gradient that would, even in the absence of Mena⁺ or Mena^{INV/+}, stimulate actin polymerization or cell protrusion activity, and measuring actin polymerization or cell protrusion activity. Both controls (1) and (2) will show actin polymerization or cell protrusion activity. Control (2) will show actin polymerization or cell protrusion activity even when the putative agent is an inhibitor of Mena⁺ or Mena^{INV/+}.

Tumor metastasis can be measured by any method known in the art including, but not limited to, an in vivo invasion assay, a modified Boyden chamber assay or capture of cells migrating into a catheterized needle. The in vivo invasion assay may be done by any method in the art including, but not limited to, insertion of a needle with EGF into the tumor, with needle collection of cells. Alternatively, tumor metastasis can be measured in vivo by any imaging method known in the art such as intravital imaging of tumors. Tumor mestatsis may also be measured by any method of imaging cells known in the art such as by high-resolution microscopy, multiphoton imaging or low resolution microscopy with staining. The tumor cells can be obtained by any method known in the art, including tumors derived from injecting a subject with cells expressing Mena⁺ or Mena^{INV/+}. Since cells expressing Mena⁺ or Mena^{INV/+} will move up an EGF gradient, when the cells expressing Mena⁺ or Mena^{INV/+} are in vivo, the cells expressing Mena⁺ or Mena^{INV/+} will move up the imposed EGF gradient and can be needle collected. If the putative agent does in fact inhibit Mena⁺ or Mena^{INV/+}, the sensitivity of cells expressing Mena⁺ or Mena^{INV/+} drops and fewer cells will move up the EGF gradient. Therefore, the collection of fewer cells Mena⁺ or Mena^{INV/+} by the in vivo invasion assay after contacting said cells with the putative agent indicates that the putative agent does in fact inhibit Mena⁺ or Mena^{INV/+}.

If a subject has a tumor expressing Mena⁺ or Mena^{INV/+}, the putative agent can be administered and metastasis of the cancer cells can be measured. Alternatively, cancer cells expressing Mena⁺ or Mena^{INV/+} can be implanted into a subject to form tumors. Mestastasis of cancer cells from these tumors may be measured by any method known in the art including in vivo assay, and measuring the number of tumor cell's in the subject's blood or lymphatic vessels.

The subject can be any mammal, such as a rodent or a human.

The present invention provides a method of treating a subject with a tumor expressing Mena⁺ or Mena^{INV/+}, the method comprising administering to the subject a Mena⁺ or Mena^{INV/+} inhibitor in an amount effective to treat the tumor.

Treating a subject's tumor means inhibiting the Mena⁺ or Mena^{INV/+} expressed by the subject's cancer cells or inhibiting the subject's tumor from metastasizing. The amount of the putative agent effective to treat the tumor will vary depending on the type of tumor, the size and severity of the tumor, and the subject's physiology. Appropriate amounts of the putative agent effective to treat the tumor can be readily determined by the skilled artisan without undue experimentation. The manner of administration of the putative agent depends on the type and site of the tumor. According to the methods of the present invention, the putative agent may be administered by any method known in the art, including but not limited to, oral or parenteral administration.

The putative agent can be a small molecule, an antibody, a peptide, a protein, a protein fragment or an aptamer. Preferably, the putative agent is hydrophilic and membrane permeable.

The tumor cell expressing Mena⁺ or Mena^{INV/+} can be a breast, pancreas, prostate, colon, brain, liver, lung, head or neck tumor cell or can be a secretory epithelial tumor cell.

The present invention provides the putative agent identified by the method for (1) determining a putative agent that binds to Mena⁺ or Mena^{INV/+}, the method comprising the steps of contacting Mena⁺ or Mena^{INV/+} and measuring bound or unbound Mena⁺ or Mena^{INV/+}; (2) determining a putative agent that inhibits Mena⁺ or Mena^{INV/+}, the method comprising the steps of contacting tumor cells expressing Mena⁺ or Mena^{INV/+} with the putative agent in the presence of a receptor tyrosine kinase-substrate gradient, and measuring actin polymerization or cell protrusion activity, wherein a decrease in or absence of actin polymerization or cell protrusion activity indicates inhibition of Mena⁺ or Mena^{INV/+}; or (3) determining a putative agent that inhibits metastasis of tumor cells expressing Mena⁺ or Mena^{INV/+} in vivo, the method comprising contacting the Mena⁺ or Mena^{INV/+} expressing tumor with the putative agent, and measuring tumor metastasis.

The present invention also provides a pharmaceutical composition comprising a Mena⁺ or Mena^{INV/+} inhibitor formulated in dosage form for treating a tumor. The formulation of the pharmaceutical composition in a dosage form for treating a tumor comprises the Mena⁺ or Mena^{INV/+} inhibitor in a pharmaceutically acceptable carrier. The pharmaceutically acceptable carrier used will depend on the method of administration as well as the subject to whom the pharmaceutical composition will be administered. Any pharmaceutically acceptable carrier known in the art can be used.

For oral administration, the formulation of the Mena⁺ or Mena^{INV/+} inhibitor may be presented as capsules, tablets, powder, granules, or as a suspension. The formulation may have conventional additives, such as lactose, mannitol, corn starch, or potato starch. The formulation may also be presented with binders, such as crystalline cellulose, cellulose derivatives, acacia, corn starch, or gelatins. Additionally, the formulation may be presented with disintegrators, such as corn starch, potato starch, or sodium carboxymethylcellulose. The formulation also may be presented with dibasic calcium phosphate anhydrous or sodium starch glycolate. Finally, the formulation may be presented with lubricants, such as talc or magnesium stearate.

For a parenteral administration, the Mena⁺ or Mena^{INV/+} inhibitor may be combined with a sterile aqueous solution which is preferably isotonic with the blood of the subject. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulations may be present in unit or multi-dose containers, such as sealed ampoules or vials. The formulation may be delivered by any mode of injection, including, without limitation, epifascial, intrasternal, intravascular, intravenous, parenchymatous, or subcutaneous.

The present invention provides for the use of a Mena⁺ or Mena^{INV/+} inhibitor for the treatment of a tumor. The present invention further provides for the use of a Mena⁺ or Mena^{INV/+} inhibitor for the preparation of a medicament for the treatment of tumor.

### REFERENCES

1. Gotswami S, Philippar U, Sun D, Patsialou A, Avraham J, Wang W, Di Modugno F, Nistico P, Gertler F B, Condeelis J S. Identification of invasion specific splice variants of the cytoskeletal protein Mena present in mammary tumor cells during invasion in vivo. Clin Exp Metastasis 2009, 26: 153-59; Epub 2008 Nov. 5.
2. Gertler F B, Niebuhr K, Reinhard M, Wehland J, Soriano P. Mena, a Relative of VASP and Drosophila Enabled, Is Implicated in the Control of Microfilament Dynamics. Cell 87: 227-239 Oct. 18, 1996.
3. Philippar U, Roussos E T, Oser M, Yamaguchi H, Kim H D, Giampieri S, Wang Y, Goswami S, Wyckoff J B, Lauffenburger D A, Sahai E, Condeelis J S, Gertler F B. A Mena Invasion Isoform Potentiates EGF-Induced Carcinoma Cell Invasion and Metastasis. Developmental Cell 15: 813-828 Dec. 9, 2008.
4. Robinson B D, Sica G L, Liu Y F, Rohan T E, Gertler F B, Condeelis J S, Jones J G. Tumor Microenvironment of Metastasis in Human Breast Carcinoma: A Potential Prognostic Marker Linked to Hematogenous Dissemination. Clin Cancer Res 2009, 15(7): 2433-41; Epub 2009 Mar. 24.
5. Condeelis J, Pollard J W. Macrophages: obligate partners for tumor cell migration, invasion, and metastasis. Cell 2006; 124:263-6.

1. A method for determining a putative agent that inhibits Mena⁺ or Mena^{INV/+}, the method comprising the steps of contacting tumor cells expressing Mena⁺ or Mena^{INV/+} with the putative agent in the presence of a receptor tyrosine kinase-substrate gradient, and measuring actin polymerization or cell protrusion activity, wherein a decrease in or absence of actin polymerization or cell protrusion activity in the presence of the agent indicates inhibition of Mena⁺ or Mene^{INV/+} and wherein a lack of a decrease in actin polymerization or cell protrusion activity indicates a lack of inhibition of Mena⁺ or Mena^{INV/+}.

2. The method of claim 1, wherein the receptor tyrosine kinase-substrate gradient is one that, in the absence of Mena⁺ or Mene^{INV/+}, would not stimulate actin polymerization or cell protrusion activity.

3. The method of claim 1, wherein the receptor tyrosine kinase-substrate is EGF.

4. The method of claim 1, comprising at least one control.

5. The method of claim 4, wherein the control comprises measuring actin polymerization or cell protrusion activity of tumor cells expressing Mena⁺ or Mene^{INV/+} in the presence of a receptor tyrosine kinase-substrate gradient.

6. The method of claim 4, wherein the control comprises contacting cells expressing Mena⁺ or Mene^{INV/+} with the putative agent in the presence of a receptor tyrosine kinase-substrate gradient which, even in the absence of Mena⁺ or Mene^{INV/+} would stimulate actin polymerization or cell protrusion activity, and measuring actin polymerization or cell protrusion activity.

7. The method of claim 1, wherein the tumor cells expressing Mena⁺ or Mene^{INV/+} are in vivo.

8. The method of claim 6, wherein measuring actin polymerization or cell protrusion activity comprises an in vivo invasion assay.

9. The method of claim 8, wherein the collection of fewer cells by the in vivo invasion assay in the tumor cells expressing Mena⁺ or Mene^{INV/+} which were contacted by the putative agent indicates that the putative agent inhibits Mena⁺ or Mena^{INV/+}.

10. A method for determining a putative agent that inhibits metastasis of a tumor, the method comprising contacting the putative agent with a cell line or tissue culture that expresses Mena⁺ or Mene^{INV/+}, wherein reduction in the expression of Mena⁺ or Mena^{INV/+} in the presence of the agent is indicative that the putative agent is a candidate for inhibiting metastasis of a tumor or wherein lack of reduction in the expression of Mena⁺ or Mene^{INV/+} is indicative that the agent is not a candidate for inhibiting metastasis of a tumor.

11. A method for determining a putative agent that inhibits metastasis of tumor cells expressing Mena⁺ or Mene^{INV/+} in vivo, the method comprising contacting the Mena⁺ or Mene^{INV/+} expressing tumor with the putative agent, and measuring tumor metastasis.

12-15. (canceled)

16. A method of treating a subject with a tumor expressing Mena⁺ or Mena^{INV/+}, the method comprising administering to the subject a Mena⁺ or Mene^{INV/+} inhibitor in an amount effective to treat the tumor.

17-19. (canceled)

20. A method for determining a putative agent that binds to Mena⁺ or Mena^{INV/+}, the method comprising the steps of contacting Mena⁺ or Mene^{INV/+} with the putative agent and measuring bound or unbound Mena⁺ or Mene^{INV/+}, wherein a increase in Mena⁺ or Mene^{INV/+} bound to the agent or a decrease in unbound Mena⁺ or Mena^{INV/+} in the presence of the agent indicates that the agent binds to Mena⁺ or Mena^{INV/+}.

21. The method of claim 1, wherein the putative agent is a small molecule, an antibody, a peptide, a protein, a protein fragment or an aptamer.

22. The method of claim 1, wherein the tumor cell expressing Mena⁺ or Mene^{INV/+} is a breast, pancreas, prostate, colon, brain, liver, lung, head or neck tumor cell.

23. The method of claim 1, wherein the tumor cell expressing Mena⁺ or Mene^{INV/+} is a secretory epithelial tumor cell.

24-26. (canceled)

27. A pharmaceutical composition comprising a Mena⁺ or Mene^{INV/+} inhibitor formulated in dosage form for treating a tumor.

28-29. (canceled)

30. A method for determining whether a subject has a metastatic tumor comprising assaying a blood, tissue and/or tumor sample of the subject for expression of Mena⁺ and/or Mena^{INV/+}, wherein overexpression of Mena⁺ and/or Mena^{INV/+} indicates the presence of a metastatic tumor.

31. The method of claim 30, which further comprises assaying the subject's blood, tissue and/or tumor sample for expression of Mena11a, wherein overexpression of Mena⁺ and/or Mena^{INV/+} and decreased expression of Mena11a together indicates the presence of a metastatic tumor.

32. A method for assessing the efficacy of therapy to treat a metastatic tumor in a subject who has undergone or is undergoing treatment for a metastatic tumor, the method comprising assaying a blood, tissue and/or tumor sample of the subject for expression of Mena⁺ and/or Mena^{INV/+}, wherein overexpression of Mena⁺ and/or Mena^{INV/+} is indicative of a need to continue therapy to treat the tumor.

33. (canceled)

34. A method for assessing the prognosis of a subject who has a metastatic tumor, comprising assaying a blood, tissue and/or tumor sample of the subject for expression of Mena⁺ and/or Mena^{INV/+}, wherein the subject's prognosis improves with a decrease in expression of Mena⁺ and/or Mena^{INV/+}.

35-39. (canceled)
